(19) United States
(12) Patent Application Publication
(10) Pub. No.: US 2023/0075608 A1
(43) Pub. Date: Mar. 9, 2023

(54) CIRCULATORY ASSIST PUMPS, ABDOMINAL BELTS FOR CHARGING CIRCULATORY ASSIST PUMPS, DEPLOYMENT CATHETERS, RETRIEVAL CATHETERS, AND RELATED SYSTEMS AND METHODS

(71) Applicant: Second Heart Assist, Inc., Salt Lake City, UT (US)

(72) Inventor: Alex Richardson, Thousand Oaks, CA (US)

(21) Appl. No.: 17/470,930

(22) Filed: Sep. 9, 2021

(51) Int. Cl. A61M 60/873; A61M 60/139; A61M 60/861; A61M 60/216; A61M 60/865; A61M 60/508; A61M 60/411; A61M 60/808; H02J 7/02; H02J 50/10; H02J 50/40; H01F 38/14

(52) U.S. Cl. CPC A61M 60/873; A61M 60/139; A61M 60/216; A61M 60/411; A61M 60/508; A61M 60/808; A61M 60/861; A61M 60/865; H01F 38/14; H02J 7/02; H02J 50/10; H02J 50/402; A61M 2205/0272; H01F 27/24

(57) ABSTRACT

A minimally invasive circulatory support platform that utilizes an aortic stent pump or pumps. The platform uses a low profile catheter-based techniques and provides temporary and chronic circulatory support depending on the needs of the patient. Further described is a wirelessly powered circulatory assist pump for providing chronic circulatory support for heart failure patients. The platform and system are relatively easy to place, have higher flow rates than existing systems, and provide improvements in the patient’s renal function.

### TECHNICAL FIELD

The application relates generally to medical devices, and more particularly to a system, apparatus, and associated methods for assisting a subject’s heart to pump blood (e.g., a circulatory assist pump).

### BACKGROUND

U.S. Pat. 8,617,239 to Reitan (Dec. 13, 2013), the contents of which are incorporated herein by this reference, relates to a catheter pump to be positioned in the ascending aorta near the aortic valve of a human being, comprising an elongated sleeve with a drive cable extending through the sleeve and connectable at its end to an external drive source and a drive rotor near the distal end of the drive cable mounted on a drive shaft being connected with the drive cable. The drive rotor consists of a propeller enclosed in a cage and the propeller and the cage are foldable from an insertion position close to the drive shaft to an expanded working position, which are characterized by means for anchoring the drive rotor in the ascending aorta near the aortic valve after insertion. Also described is a method to position the pump of a catheter pump in the ascending aorta just above the aortic valve.

U.S. Pat. 8,617,239 to Reitan builds upon an earlier patent of Reitan, i.e., U.S. Pat. 5,749,855 to Reitan (May 12, 1998), the contents of which are also incorporated herein by this reference, which relates to a drive cable, with one end of the drive cable being connectable to a drive source and a collapsible drive propeller at the other end of the drive cable. The collapsible drive propeller is adjustable between a closed configuration in which the collapsible drive propeller is collapsed on the drive cable and an open configuration in which the collapsible drive propeller is expanded so as to be operative as an impeller. A sleeve extends between one side of the collapsible drive propeller and the other side of the collapsible drive propeller with the sleeve being movable between configurations in which the collapsible drive propeller is in the open and closed configuration. A lattice cage is arranged surrounding the propeller and is folded out at the same time as the propeller. As described by U.S. Pat. 8,617,239 to Reitan, while the device of U.S. Pat. 5,749,855 operates very well in many circumstances, there is still room for improvement.

An even earlier blood pumping catheter is described in U.S. Pat. 4,753,221 to Kensey et al. (Jun. 28, 1988), the contents of which are incorporated herein by this reference. Kensey et al. relates to an elongated catheter for pumping blood through at least a portion of a subject’s vascular system. The catheter is of a sufficiently small diameter and flexibility to enable it to be passed through the vascular system so that the distal end portion of the catheter is located within or adjacent the patient’s heart. A rotatable pump is located at the distal end of the catheter and is rotated by drive means in the catheter. The distal end portion of the catheter includes an inlet for blood to flow therein and an outlet for blood to flow therefrom. The catheter is arranged so that blood is pumped by the catheter’s pump through the heart and into the vascular system without requiring any pumping action of the heart.

Other catheter pumps are known from U.S. 2008/0132748 A1, U.S. 2008/0114339A1, and WO03/103745A2, the contents of each of which are incorporated herein by this reference.

### BRIEF SUMMARY

Described, among other things herein, is a minimally invasive circulatory support platform that utilizes an aortic stent pump. The platform uses a low profile, catheter-based technique and can be used to provide temporary and/or chronic circulatory support depending on the needs of the subject or patient (e.g., a mammal, such as a human).

In certain embodiments, the described device may include a battery (or electrical storage device) powered circulatory assist pump (or pumps) positioned within an aortic stent which may be wirelessly charged with an abdominal belt.

The described platform and system are relatively easy to place, have higher flow rates than existing systems, and provide improvements in a patient’s renal function. The chronic circulatory assist device (which is removable) is placed within an aortic stent that is preferably wirelessly powered. The impeller is shaped and designed to maximize safety and blood flow and to reduce the risk of hemolysis.

In use, the catheter may be introduced “percutaneously” into the lower aorta via, e.g., the normal “Seldinger technique” in the groin (a small incision into the femoral artery) and fed up to the aorta to the desired position (e.g., the descending aorta). The pump may be inserted in the groin area and introduced into the femoral artery (e.g., to just above the renal arteries in the descending aorta) with the help of a small surgical insertion and insertion sheath. The pump is thereafter fed up into the desired position in the lower aorta.

Alternatively, the pump may be placed via axillary entry in the neck or chest of the subject. See, e.g., K M. Doersch “Temporary Left Ventricular Assist Device Through an Axillary Access is a Promising Approach to Improve Outcomes in Refractory Cardiogenic Shock Patients,” ASAIO J. 2015 May-Jun; 61(3): 253-258; doi: 10.1097/MAT.0000000000000222, the contents of which are incorporated herein by this reference, which describes implantation of a temporary left ventricular assist device (“LVAD”) through an axillary approach as a way to provide adequate circulation to the patient, avoid multiple chest entries and infection risks.

In some embodiments, a battery and motor are utilized to drive the pump. An external belt may be provided that wirelessly charges the battery.

The external belt or vest (electric powered coil inside that extends along the length of the belt such that the coil surrounds the patient’s abdomen when worn) and appropriate circuitry, which belt or vest provides an electromagnetic field. For example, a transmitting coil associated with the belt or vest transmits AC energy, which is received by a receiving coil associated with the wireless pump, which DC energy can be used to power a motor (e.g., pump) and/or a battery. In certain embodiments, the system is controlled, e.g., by a watch (not shown) connected wirelessly to the belt, vest, or controller.

Also described are methods for providing circulatory assist to a subject in need thereof, the method comprising: using the described systems to provide circulatory assist to the subject. Such methods include methods where a “puckless” TET is positioned within a patient’s vasculature such as within the aorta, including the descending aorta.

### BRIEF DESCRIPTION OF THE DRAWINGS

FIG. 1 depicts a lobe design according to the instant disclosure displaying deployed (or extended) arm-like impeller blades.

FIG. 2 depicts the lobe design of FIG. 1 displaying retracted arm-like impeller blades.

FIG. 3 depicts a front view of the lobe design of FIG. 2 displaying deployed (operational) impellers.

FIG. 4 is a cross-sectional view of the device of FIG. 1.

FIG. 5 depicts a wireless circulatory assist pump according to an embodiment of the disclosure.

FIG. 6 depicts a belt and controller.

FIG. 7 depicts an induction coil assembly of a wireless charging circuit according to an embodiment of the disclosure.

FIG. 8 depicts the tip of a deployment catheter according to an embodiment of the disclosure for the deployment of a wireless circulatory assist pump.

FIG. 9 depicts the tip of a retrieval catheter according to an embodiment of the disclosure for the removal of a wireless circulatory assist pump.

FIG. 10 depicts a wireless pump and separated placement/retrieval catheter according to embodiments of the disclosure.

FIG. 11 depicts a wireless pump with connected placement/retrieval catheter according to embodiments of the disclosure.

FIGS. 12-14 depict a deployment procedure for placing the wireless pump.

FIGS. 15-18 depict a procedure for retrieving the wireless pump.

FIGS. 19 and 20 depict a further embodiment of the wireless pump, which utilizes two sets of impeller blades, which design adds flow to the low RPM (< 4 K) MCAD system.

### DETAILED DESCRIPTION

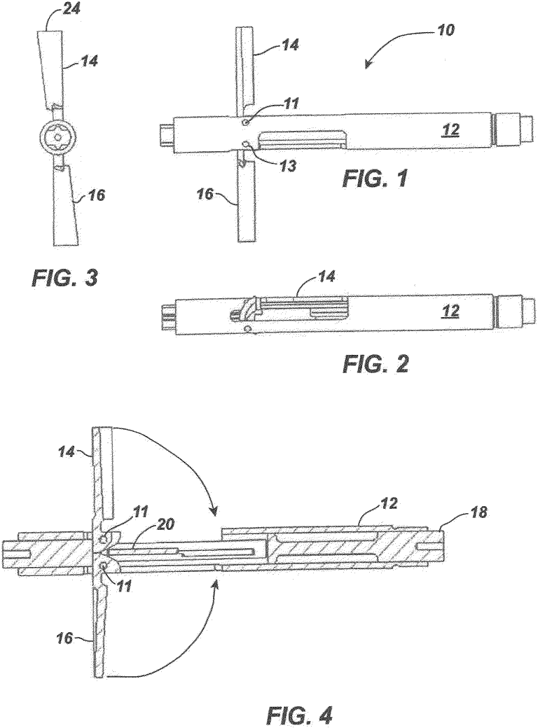

An aspect of the disclosure is a circulatory assist pump, generally 10, shown in FIGS. 1, 3, and 4 in its operational position. The circulatory assist pump 10 comprises a tubular elongated casing 12 associated with a pair of arm-like impeller blades 14, 16. The depicted impeller blades are pivotally associated with the remainder of the lobe by pivots (e.g., pins or shafts) 11 placed in apertures 13 in the tubular elongated casing 12 and impeller blades. The impeller blades are outwardly foldable and retractable, and can move, e.g., into a position perpendicular to the tubular elongated casing 12. As can be determined, the accompanying figure drawings are generally not drawn to scale.

The depicted circulatory assist pump includes a positioning cable 18 running along the impeller axis, about which the impeller blades 14, 16 (along with the rest of the device) rotate to create a pump action, for example, in the aorta. The arm-like nature of the depicted blades allows them to extend maximally from the remainder of the body when in a perpendicular position and fill a large portion of the descending aorta. At the end of the positioning cable is a rod 20 that interacts with a cam portion 22 of each impeller blade 14, 16. Advancing (or relatively displacing) the rod 20 so that it abuts and actuates the cam portion 22 causes the withdrawn impeller (FIG. 3) to extend outwards from the rest of the lobe (FIG. 1). The cam lobe design (FIG. 4) is utilized to expand and retrieve the impeller into and out of the catheter, which is far more reliable deployment than with, for example, a spring design, although a spring may also be used herein. For example, springs vary with temperature and manufacturing, while cam lobes are consistent and remain constant. In certain embodiments, the impeller blades can be tilted on demand (in the same manner as the way an airplane wing flaps are controlled) by, e.g., adjustment of the cams, which balances hemolysis, thrust, and flow.

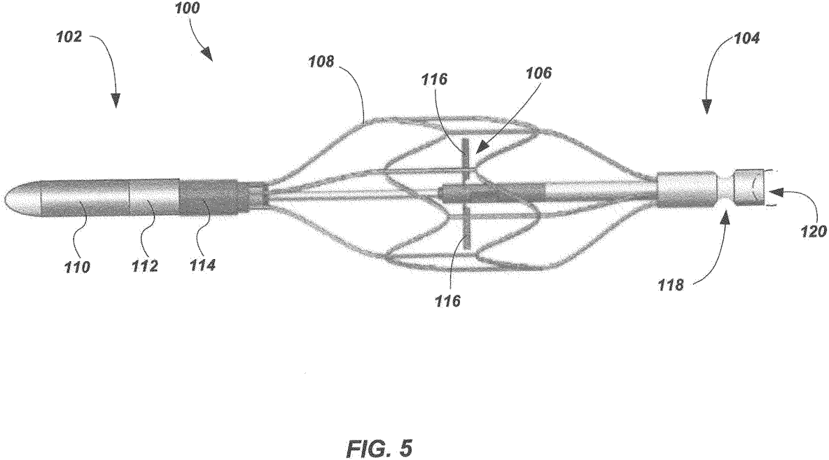

An aortic stent cage surrounds the impeller (see, e.g., FIG. 5) and preferably has the most open area possible, so as to reduce hemolysis.

The pump may be placed, for example, above the renal arteries in the aorta to aid in kidney function. More flow into the kidneys means more rapid removal of excess fluids, which leads to better revival of kidney function. In certain embodiments, the system preferably uses the full diameter of the aorta to increase pump stability and reduce pump migration.

In certain embodiments, the system includes implanted sensors that assist with a real time, automatic adjustment and management of the circulatory assist support system based upon data provided by the implanted (preferably wireless) sensors. The sensors monitor fluid flow and provide feedback and data to the system, which feedback and data is used to, e.g., adjust the speed and/or angle of the impeller to increase or decrease fluid flow and pressure.

The wireless power embodiment is designed to reduce infection risk compared to external drive line systems. Also, the wireless power option helps improve the patient’s quality of life.

Optionally, the system may be utilized with an upper aorta pulsating aortic cuff stent graft, which improves the total flow of the system, improves hemodynamics, (via the pulsatile flow) improves the release of beneficial proteins for organ health, and reduces RPMs needed by the impeller to reach desired flow rates.

In certain embodiments, elements of a system and/or device as described herein (e.g., impeller blade(s), drive shaft, and/or stent cage) are coated with a hydrophobic or lubricous material to reduce the potential for endothelialization after placement of the device. Such a material can be, for example, expanded polytetrafluorethylene (ePTFE available from Gore Technologies) or similar graft liner.

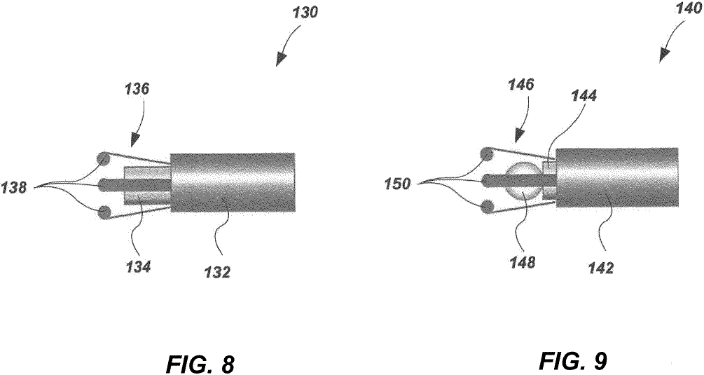

In some embodiments, such as shown in FIG. 5, a wireless circulatory assist pump 100 may be configured to be deployed into and removed from a vein or artery (e.g., the aorta) via one or more catheters (FIGS. 8-9). In certain embodiments, a spring or retainer clip on the catheter is used to secure the deployment and removal process with the catheter(s). In certain embodiments, the clips are typically made of nitinol. The wireless circulatory assist pump 100 may comprise a distal tip end 102, a proximal docking end 104, and an impeller 106 enclosed within a stent cage 108 therebetween. The wireless circulatory assist pump 100 may further comprise a battery 110, circuitry 112, and a motor 114. The circuitry 112 may comprise a wireless charging circuit, a communications circuit, and a control circuit. As shown in FIG. 5, the battery 110, the circuitry 112, and the motor 114, may all be located at or near the distal tip end 102, but it will be understood that one or more, or all, of the battery 110, the wireless charging circuit, the communications circuit, the control circuit, and the motor 114, may alternatively be located at or near the proximal docking end 104.

The stent cage 108 may be configured to securely position the wireless circulatory assist pump 100 in a patient’s aorta, while maintaining the pulsatility of the aorta. Additionally, the stent cage 108 may be compressed and stowed for placement and removal of the wireless circulatory assist device 100.

The motor 114 may be a miniature brushless direct current (“DC” or “BLDC”) motor. For example, the motor 114 may be a miniature brushless DC motor such as available under the tradename “EC6” from Maxon Precision Motors, Inc. of Foster City, California US. A BLDC motor, electronically commutated motor (ECM or EC motor) or synchronous DC motor, is a synchronous motor using a direct current (DC) electric power supply. An ASIC may be used to control BLDC motor and telemetry in the mechanical circulatory assist device system.

The battery 110 may be a rechargeable battery, such as a lithium-ion battery. For example, the battery 110 may be a 3 milliamp hour (mAh) lithium-ion battery available under the tradename “CONTIGO” from EaglePicher Technologies of Joplin, Missouri USA. For another example, the battery 110 may be a 3 mAh lithium-ion battery available under the tradename “MICRO3-QL0003B” from Quallion LLC of Sylmar, California USA. It will be understood, however, that the battery 110 may be of any suitable chemistry and/or type, including non-chemical electric power storage devices, such as a capacitor (e.g., a supercapacitor, ultracapacitor, or double-layer capacitor).

The wireless charging circuit may produce an electric current in response to an applied electric field, magnetic field, and/or electromagnetic field, which may be utilized to charge the battery 110. Additionally, the wireless charging circuit may include an induction coil assembly, which will be further described with reference to FIG. 7.

The communication circuit may be configured to send and receive data via wireless communication. For example, the communication circuit may be configured to send and receive data utilizing radio communication (e.g., WiFi, Bluetooth, etc.) In some embodiments, the communication circuit may be utilized to send data collected from one or more sensors of the wireless circulatory assist pump 100. For example, the communication circuit may be utilized to send data relating to the rotational speed of the pump, upstream and downstream fluid pressures, battery charge status, motor status, impeller status, and/or other measured conditions.

The control circuit may be utilized to control certain operations of the wireless circulatory assist pump 100. In some embodiments, the control circuit may be utilized to control the rotational speed of the motor 114, the shape of the impeller 106, the deployment of the impeller blades 116, the stowing of the impeller blades 116, the angle of the impeller blades 116, and/or other operations of the circulatory assist pump 100.

In some embodiments, the circulatory assist pump 100 may comprise one or more application-specific integrated circuit (“ASIC”) chips. For example, one or more of the charging circuit, the communication circuit, and the control circuit may be provided as one or more ASIC chips.

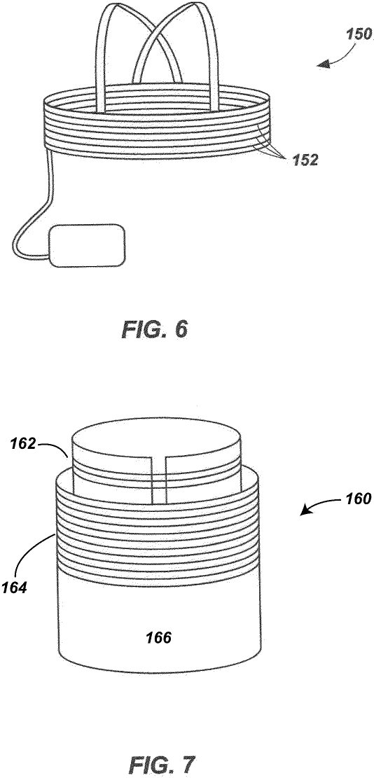

Electromagnetic waves may be delivered non-invasively from an abdominal belt 150, as shown in FIG. 6, to a wireless charging circuit. The abdominal belt 150 may comprise a coil 152 of wire that extends around the entire circumference of the abdominal belt 150. Accordingly, when the abdominal belt 150 is worn by a patient, the coil 152 may extend circumferentially around the abdomen of the patient.

Once fitted onto the patient, the abdominal belt 150 (or vest, not shown) is typically configured to deliver electromagnetic waves at a relatively low frequency (e.g., below gigahertz, and preferably below megahertz) using a flux field. The particular frequency transmitted will be chosen based upon, e.g., the number of lining(s), windings of coil(s), and the type and mass of materials used in the particular wireless pump 100. For example, the abdominal belt 150 may be configured to deliver electromagnetic waves at a frequency between about 50 kHz and about 300 kHz. For another example, the abdominal belt 150 may be configured to deliver electromagnetic waves at a frequency between about 100 kHz and about 150 kHz. In yet another example, the abdominal belt 150 may be configured to deliver electromagnetic waves at a frequency of about 125.3 kHz. Preferably, the frequency will be about (within 25% of) 123.5 kHz, which greatly reduce SAR (i.e., below AM and close to audible) and result in less heating, with more efficient and easier energy transfer to the motor and/or a battery associated with the wireless pump.

As shown in FIG. 7, the wireless charging circuit of the wireless circulatory assist pump 100 (FIG. 5) may comprise an induction coil assembly 160 and energy may be transferred to the wireless charging circuit from the abdominal belt 150 via inductive coupling. The induction coil assembly 160 may comprise a dual-coil receiver comprising a first coil 162 and a second coil 164, the first and second coils 162, 164 being separated by a dielectric material (e.g., an air gap). The first coil 162 may be positioned to be exposed to an applied electromagnetic field, such as generated by the abdominal belt 150. For example, at least a portion of the first coil 162 may be positioned within a portion of an enclosure that is transparent to electromagnetic fields. Meanwhile, the second coil 164 may be shielded from the electromagnetic fields generated by the abdominal belt 150. At least a portion of the first coil 162 and the second coil 164 may be positioned around a core 166, which may be magnetic. A current may be induced in the first coil 162 by the applied electromagnetic field from the abdominal belt 150. In turn, the current flowing through the first coil 162 may generate an electric field that is directed through the second coil 164 via the core 166, which may induce an electric current in the second coil 164. The electric current induced in the second coil may be directed to load requirements of the circulatory assist pump 100, such as charging the battery and/or powering the motor. By using such a dual-coil receiver configuration, the impedance of the induction coil assembly 160 may be reduced compared to a single coil receiver. Additionally, the load (e.g., charging of the battery, powering the motor, etc.) may be decoupled from the first coil 162, which may improve performance. For example, the power may be supplied at a relatively consistent rate from the abdominal belt 150, while the load demand may fluctuate.

Delivering the electromagnetic energy from the coils 152 surrounding the entire circumference of a patient’s abdomen to the wireless charging circuit at a relatively low frequency may have many advantages over traditional transcutaneous energy transmission (“TET”) systems that utilize a relatively small puck (e.g., having a diameter between about 2-3 inches) that delivers energy at a relatively high frequency. First, the distribution of relatively low frequency electromagnetic waves over a relatively large area, may reduce the heating of body tissue when compared to a relatively small puck that delivers energy at a relatively high frequency. Second, the distribution of relatively low frequency electromagnetic waves over a relatively large area may improve the reliability and range of the energy delivery to the wireless charging circuit of the implanted device. The relatively low frequency electromagnetic waves may travel more efficiently through relatively dense materials, such as body tissue. Accordingly, while small puck devices that deliver relatively high frequency electromagnetic energy may require precise alignment to reliably deliver energy, the wireless charging circuit and the abdominal belt 150 delivering relatively low frequency electromagnetic waves may be rotated up to 45 degrees relative to one another and reliable energy transfer may still occur.

Referring again to FIG. 5, the impeller 106 may be configured to change shapes in one or more various ways. The impeller 106 may comprise impeller blades 116 that may be configured to move between a deployed position, as shown, and a stowed position (see FIG. 2). The impeller blades 116 may additionally be configured to move to positions between the deployed position and the stowed position (e.g., a partially deployed position).

In some embodiments, the impeller blades 116 may be configured to rotate or twist to selectively vary the pitch of the impeller blades 116. In some embodiments, the impeller blades 116 may be configured to bend to selectively alter the curvature of the impeller blades 116.

Certain impeller shapes and curvatures can optimize blood flow and minimize hemolysis in both chronic implantable and temporary circulatory assist devices. Most of these ideal optimized shapes, however, are not practical for delivery via a percutaneous non-surgical delivery catheter. Additionally, not one impeller shape appears to be ideal for all circumstances to best meet patient needs at all times. Accordingly, impellers 106 according to embodiments of the disclosure may change shape on demand to meet patient needs as they arise that can be delivered and removed without surgery. Traditionally, these ideal impeller shapes are fixed in shape and cannot be changed without mechanically making a change in manufacturing.

As previously discussed, the distal tip end 102 of the circulatory assist pump 100 may house the battery 110, the wireless charging circuit, the communications circuit, the control circuit, and the motor 114. The end of the distal tip end 102 may have a smooth, generally dome shaped, leading end. This may prevent harm to the patient should the distal tip end 102 come into contact with the arterial wall, such as during an insertion or removal procedure. The distal tip end 102 may comprise a canister covering and sealing the components therein. In some embodiments, a titanium canister may cover and seal the distal tip end 102.

In yet additional embodiments, the canister may comprise at least a portion that is made of a material that is transparent to certain frequencies of electromagnetic radiation, magnetic fields, and/or electrical fields, such as a ceramic (“sealed ceramic”) or a polymer, to facilitate electromagnetic, electric, and/or magnetic communication between devices located outside of the patient’s body (e.g., the abdominal belt 150) and components within the distal tip end 102 (e.g., the first coil 162 of the charging circuit). For example, the use of a ceramic fused to the titanium canister provides for radio frequency (RF) transparency or translucency.

The proximal docking end 104 of the circulatory assist pump 100 may comprise features configured to interact with one or more catheter, such as a deployment catheter 130 (FIG. 8) and a retrieval catheter 140 (FIG. 9). In some embodiments, the proximal docking end 104 may comprise an annular groove 118 located proximal to an end surface of the proximal docking end 104. The end surface may be dished to provide a generally hemispherical indentation 120 in the proximal docking end 104. In some embodiments, the proximal docking end 104 may comprise a ferromagnetic material.

In certain embodiments described herein, a self-aligning magnetic design is utilized for the device docking and retrieval catheter(s).

To install the circulatory assist pump 100 a deployment catheter 130 may be provided having a tip configured to hold and then release the circulatory assist pump 100, as shown in FIG. 8. As shown, the deployment catheter 130 may comprise an outer sheath 132, an inner member 134, and a plurality of fingers 136 located between the outer sheath 132 and the inner member 134.

When the inner member 134 and the fingers 136 are extended out of the outer sheath 132, the tips of the fingers 136 may be biased radially outward and apart from one another. Each finger may comprise a protrusion 138 at the tip, which may be spaced sufficiently apart that the proximal docking end 104 may freely pass between the protrusions 138. Accordingly, the proximal docking end 104 may be positioned adjacent to the inner member 134, and the protrusions 138 may surround the annular groove 118 of the proximal docking end 104.

The outer sheath 132 may then be extended over the inner member 134 and the plurality of fingers 136. As the outer sheath 132 extends over the fingers 136, the outer sheath 132 may force the tips of the fingers 136 radially inward and the protrusions 138 of the fingers 136 may be positioned within the annular groove 118 of the proximal docking end 104 of the circulatory assist pump 100, and prevent movement of the proximal docking end 104 relative to the inner member 134 and the fingers 136. The blades 116 of the impeller 106 may be placed into a stowed position and the stent cage 108 may be retracted. In some embodiments the blades of the impeller 116 and the stent cage 108 may be withdrawn into the outer sheath 132. For example, embodiments that utilize a shape change impeller 106A, may have impeller blades 116A with sufficient flexibility that the impeller blades 116A may naturally fold and conform as the impeller blades 116A are withdrawn into the outer sheath 132.

The tip of the deployment catheter 130 and the attached circulatory assist pump 100 may then be positioned within a patient to a desired location for deployment of the circulatory assist pump 100. The resilient material of the stent cage 108 may expand to contact the patient’s vessel wall and hold the circulatory assist pump 100 in place. Then, the outer sheath 132 may be withdrawn from the fingers 136 and the inner member 134. As the outer sheath 132 is withdrawn, the tips of the fingers 136 may be biased radially apart and the protrusions 138 of the fingers 136 may be withdrawn from the annular groove 118, disconnecting the deployment catheter 130 from the proximal docking end 104. The deployment catheter 130 may then be removed from the patient with the circulatory assist pump 100 left in place.

To remove the circulatory assist pump 100, a retrieval catheter 140 having a tip such as shown in FIG. 9 may be utilized. The retrieval catheter 140 may be generally similar to the deployment catheter 130, having an outer sheath 142, an inner member 144, and a plurality of fingers 146 located between the outer sheath 142 and the inner member 144. The retrieval catheter 140, however, may additionally include a magnetic ball 148 (or ring magnet; not shown) positioned at the end of the inner member 144.

When the inner member 144 and the fingers 146 are extended out of the outer sheath 142, the tips of the fingers 146 may be biased radially outward and apart from one another. Each finger 146 may comprise a protrusion 150 at the tip, which may be spaced sufficiently apart that the proximal docking end 104 may freely pass between the protrusions 150. Accordingly, the proximal docking end 104 may be positioned adjacent to the inner member 134, and the magnetic ball 148 may be attracted to the ferromagnetic material of the proximal docking end 104 and become seated within the generally hemispherical indentation 120 in the proximal docking end 104 and magnetically coupled thereto. Upon the seating and magnetic coupling of the magnetic ball 148 to the proximal docking end 104, the protrusions 150 may surround the annular groove 118 of the proximal docking end 104.

The outer sheath 142 may then be extended over the inner member 144 and the plurality of fingers 146. As the outer sheath 142 extends over the fingers 146, the outer sheath 142 may force the tips of the fingers 146 radially inward and the protrusions 150 of the fingers 146 may be positioned within the annular groove 118 of the proximal docking end 104 of the circulatory assist pump 100, and prevent movement of the proximal docking end 104 relative to the inner member 144 and the fingers 146. The blades 116 of the impeller 106 may be placed into a stowed position and the stent cage 108 may be retracted from the artery wall. In some embodiments the blades of the impeller 116 and the stent cage 108 may be withdrawn into the outer sheath 142. For example, embodiments that utilize a shape change impeller 106A, may have impeller blades 116A with sufficient flexibility that the impeller blades 116A may naturally fold and conform as the impeller blades 116A are withdrawn into the outer sheath 142. The retrieval catheter 140 and circulatory assist pump 100 may then be removed from the patient.

Also described herein is chronic, wireless mechanical circulatory assist device (MCAD) and system, which may be percutaneously placed into the descending thoracic aorta above the kidneys of a subject with, for instance, the aid of placement and retrieval catheters (e.g., 14 Fr), and utilized in the treatment of, for example, heart failure and associated renal dysfunction.

In a preferred embodiment, the wireless MCAD system includes an aortic wireless pump, an appropriately sized wearable vest or belt for providing energy to drive the wireless pump, while the wireless pump is powered and controlled by a, e.g., radio frequency (RF) control unit with power pack.

The aortic wireless pump in such a preferred system typically includes a radial force nitinol stent cage, which expands up to, e.g., 22 mm in deployment in an adult male. Such a size for the wireless pump allows for secure placement in the aorta, while allowing the aorta to maintain its puslatility. Aortic pulsatility is ideal for extended use applications.

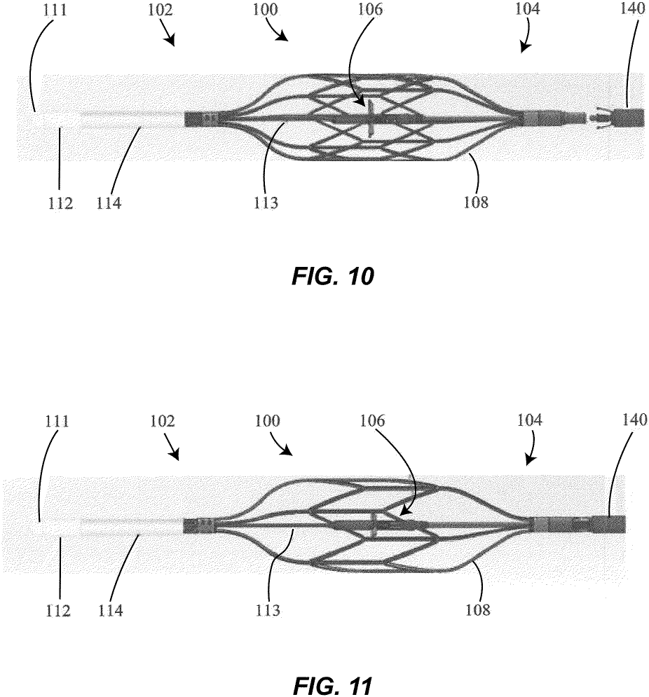

A presently preferred wireless pump is depicted, e.g., in FIGS. 10 and 11. The pump may be placed with the aid of a placement/retrieval catheter. In FIG. 10, for instance the catheter is disconnected from the wireless pump, while in FIG. 11, it is shown connected.

The wireless pump 100 depicted in FIGS. 10 and 11 is configured to be deployed into and removed from an artery (e.g., the aorta) via one or more catheters 140. In certain embodiments, a spring clip may be used on the catheter to secure deployment and removal. The depicted wireless pump 100 has a distal tip end 102, a proximal docking end 104, and an impeller 106 enclosed within a radial force stent cage 108 made of a memory shape material such as nitinol. The depicted wireless pump 100 further includes circuitry 112, such as a battery and/or motor 114. The circuitry 112 of FIGS. 10 and 11 is typically sealed in an RF translucent material such as ceramic, ePTFE, or Polyether ether ketone (“PEEK”) (so as not to block RF), and may comprise a wireless charging circuit (e.g., an inductive charging coil), a communications circuit, and a control circuit. Although a “puck” receiving coil might be utilized, a “puckless” TET is preferably used, which coil comprises a high permeability, ferromagnetic core with a high Q RF coil (miniaturized receiving coil) positionable inside of the distal tip of the wireless pump. See, e.g., Loeb et al. “Bion system for distributed neural prosthetic interfaces” Medical Engineering and Physics, 23(1):9-18, January 2001; U.S. Pat. 7,005,935 to Moore (Feb. 28, 20206) for “Switched reactance modulated E-class oscillator”.

The motor and/or battery can be placed within a sealed canister (e.g., titanium or stainless steel).

Placement of the circuitry, motor, and battery near the distal tip causes the wireless pump to have its primary weight at the top of the device, which helps to mitigate positional movement by impact (e.g., jumping). This configuration also increases the radial force of the stent cage of the wireless pump.

The radial force stent cage 108 is configured to securely position the wireless pump 100 in a particular patient’s aorta, while maintaining the aorta’s pulsatility. Additionally, the stent cage 108 may be compressed and stowed for placement and removal of the wireless circulatory assist device 100.

The motor 114 is preferably a miniature brushless direct current (“DC” or “BLDC”) motor. A BLDC motor, electronically commutated motor (ECM or EC motor) or synchronous DC motor, is a synchronous motor using a direct current (DC) electric power supply. Such a brushless motor preferably has a relatively small diameter (e.g., < 14 Fr), but the diameter could be larger dependent on the application. An ASIC may be used to control BLDC motor and telemetry in the mechanical circulatory assist device system. A drive shaft 113 connects the motor to the impeller 106.

Percutaneous placement of the wireless pump using the placement/retrieval catheter (by, e.g., femoral arterial access) can be surgically accomplished in about two minutes.

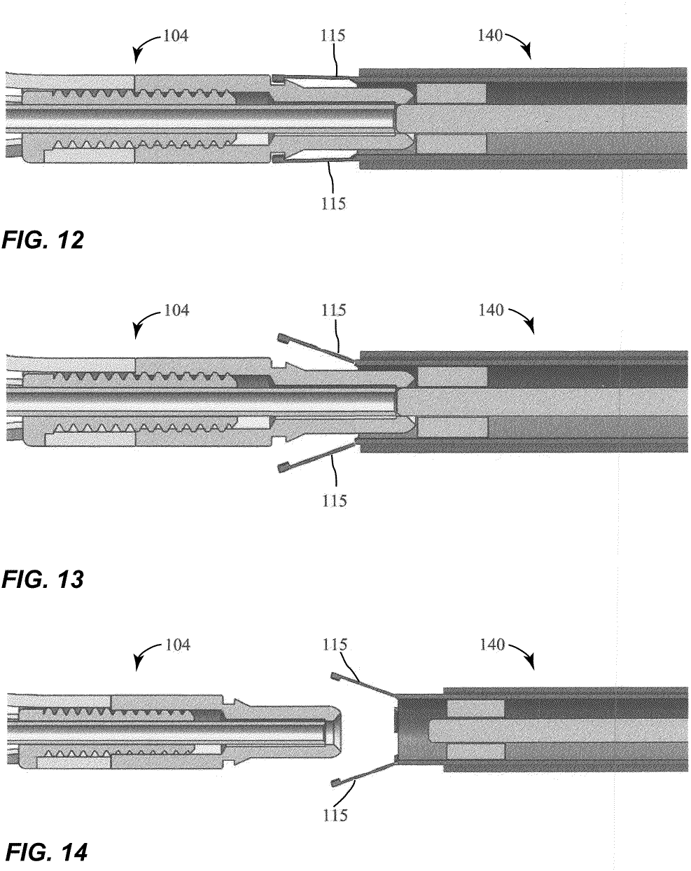

FIGS. 12-14 depict a deployment procedure for placing the wireless pump in the subject. In FIG. 12, the distal tip end of the wireless pump is placed in, for example, the descending thoracic aorta above the subject’s kidneys utilizing a deployment/removal catheter 140. The catheter for placement preferably does not have a magnetic tip. A set of clips 115 (or capture clasps) that interact with an indented feature on the proximal docking end 104 of the wireless pump attach the catheter 140 to the wireless pump. Once properly positioned in the aorta, the clips 115 are opened (FIGS. 13 and 14) and disengage the wireless pump. The catheter is then withdrawn (FIG. 14) from the proximal end 104 of the catheter 140, and the catheter removed from the subject’s circulatory system.

Once properly placed in the subject’s aorta, there is typically no need for repositioning of the wireless aortic pump, which alleviates the need for hooks or paddles that might damage the wall of the aorta.

Further, the aortic wireless pump utilizes a relatively low speed and is shown to have ultra-low hemolysis. The speed of the aortic wireless pump (in RPM) in certain embodiments is set at a chosen RPM. In certain embodiments, however, closed loop feedback and/or differential pressure management is used to adjust the RPM of the wireless pump appropriately.

Wireless transcutaneous energy transfer (TET) is preferably used to operate and provide power to the wireless pump 100 (i.e., without utilizing a separate “puck” or separate subcutaneous receiving (Rx) coil device). See, e.g., G. E. Loeb, R. A. Peck, W. H. Moore, and K. Hood. Bion system for distributed nerual prosthetic interfaces. Medical Engineering and Physics, 23(1):9-18, January 2001; and U.S. Pat. 7,005,935 to Moore (Feb. 28, 2006) for “Switched reactance modulated E-class oscillator”. The resulting lack of wires in the aorta and/or femoral arteries reduces the risk of infection.

Preferably, ultralow power magnetic induction power transmission is used to power and control such a wireless pump. This results in truly wireless operation, with little to no heat generation. The system utilizes a low frequency design with extremely conservative operating frequencies and extraordinarily low specific absorption rate (SAR) value. SAR is a measure of the rate at which energy is absorbed per unit mass by a human body when exposed to a radio frequency (RF) electromagnetic field. It is defined as the power absorbed per mass of tissue and has units of watts per kilogram (W/kg).

The wireless pump is further preferably configured so that the magnetic flux (or flux area) of the wireless pump accommodates angles of anatomical variability so that the device maintains constant operation.

In certain embodiments, the wireless pump further includes a sensor 111 or sensors in one or more ends of the wireless pump (e.g., at the end 102, proximal 112 of FIG. 10) to measure and preferably report, e.g., blood pressure. Brancato, Luigi et al. “An Implantable Intravascular Pressure Sensor for a Ventricular Assist Device.” Micromachines vol. 7,8 135. 8 Aug. 2016, doi:10.3390/mi7080135. Control of the sensor(s) can be via the ASIC. In such embodiments, the MCAD system can provide auto correction / selection of speed through the internal feedback of the sensors. Built in telemetry can also be used for communications.

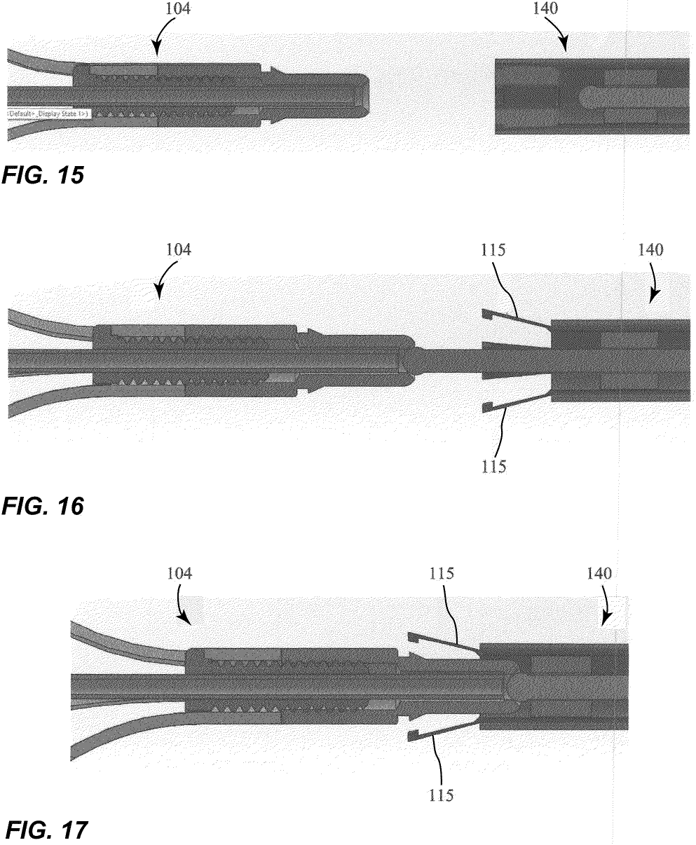

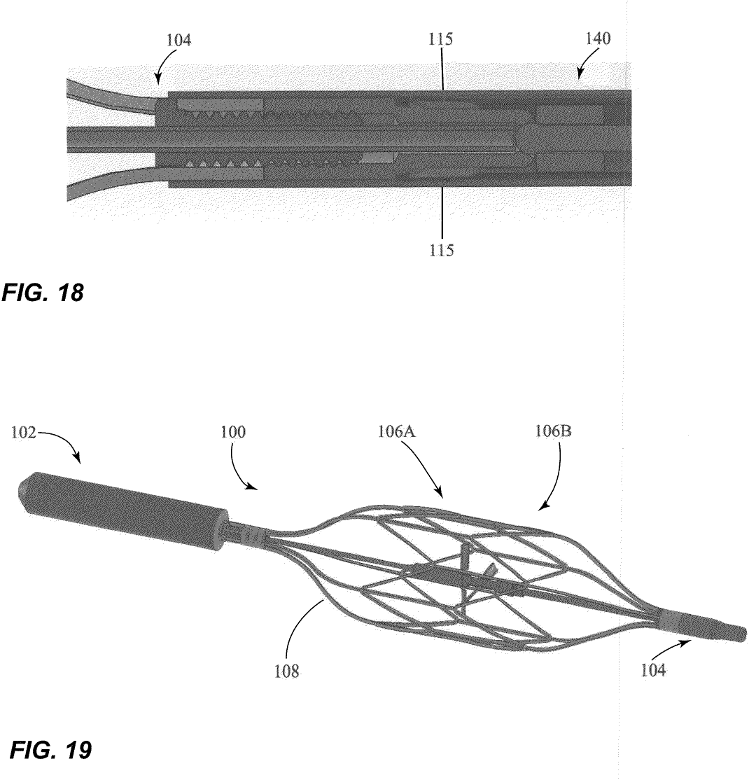

After placement, the wireless pump can be removed by generally reversing the deployment procedure (see, e.g., FIGS. 15 - 18). In a removal procedure, a magnetic ball alignment tip (FIG. 9) or magnetic ring (not shown) is preferably present at the tip of the catheter 140 to interact with a ferromagnetic material present on the proximal docking end 104 of the wireless pump. The magnetic ball alignment tip (or ring magnet; not shown) provides for a self-aligning magnetic design for the device docking and retrieval catheter. The catheter typically enters the subject’s body via the femoral artery near the subject’s groin and is advanced proximal the placed wireless pump. A femoral puncture is a minimally invasive method of placing the device. In FIG. 15, the wireless pump and the removal catheter are shown separated. FIG. 16 depicts an “inner stage” where the magnetic tip has been extended from the stent cage retracting sheath of the catheter and placed clips 115 have been opened for interaction with the wireless pump. In FIG. 17, an “inner connected” stage is depicted, wherein the wireless pump is shown being drawn back into the stent cage retracting sheath of the catheter. When appropriately placed, the clips 115 grasp the proximal tip end 102 of the wireless pump and interact with appropriately shaped notches or grooves formed on the sides of the distal docking end. The wireless pump can then be further drawn back into the stent cage retracting sheath (FIG. 18). Eventually, the nitinol stent cage too is collapsed and stored within the sheath of the catheter.

The RF control unit (with power pack) of the MCAD system preferably operates for at least 36 hours on a primary rechargeable battery. The RF control unit is also preferably provided with an indicator and a secondary battery that adds an additional 36 hours of operation. U.S. Pat. 7,177,690 to Woods et al. (Feb. 13, 2007) for “Implantable system having rechargeable battery indicator”. The secondary battery is readily swappable with the primary rechargeable battery to prevent a disruption of operation.

In certain embodiments, the RF control unit is constructed to be splash proof/water resistant to 3 ATM (30 m). In certain embodiments, the RF control unit may also be relatively lightweight and readily mounted onto a belt or integrated pouch on the vest of the MCAD system.

In certain preferred embodiments, the RF control unit is Bluetooth-enabled so as to provide functional support and status, including providing information to a health care professional.

The vest of the MCAD system is preferably lightweight and flexible, having a breathable / washable fabric outer shell. The vest is typically made of cloth, having straps and, e.g., Velcro ® adjustments. The vest preferably has an accessible front connector for easy connection or detachment. It is further also preferably constructed to be splash proof/water resistant.

The MCAD system, once properly placed and implemented, augments native flow through the aorta with as much as an additional four (4) liters of blood per minute.

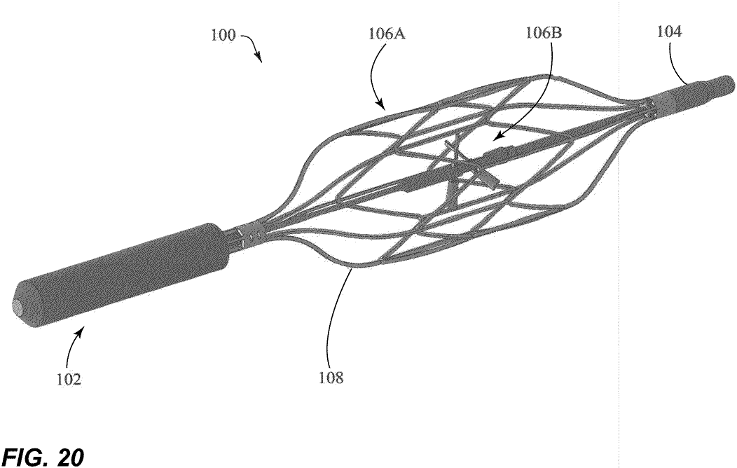

FIGS. 19 and 20 depict a further embodiment of the wireless pump, which utilizes two sets of impeller blades. In such an embodiment, a second set of impeller blades is positioned further along the drive shaft from the first set. The added impellers increase blood flow without needing to increase the low RPM (< 4 K) of the MCAD system.

While certain illustrative embodiments have been described in connection with the figures, those of ordinary skill in the art will recognize and appreciate that the scope of this disclosure is not limited to those embodiments explicitly shown and described in this disclosure. Rather, many additions, deletions, and modifications to the embodiments described in this disclosure may be made to produce embodiments within the scope of this disclosure, such as those specifically claimed, including legal equivalents. In addition, features from one disclosed embodiment may be combined with features of another disclosed embodiment while still being within the scope of this disclosure.

### References

(the contents of each of which are incorporated herein by this reference.)

Brancato, Luigi et al. “An Implantable Intravascular Pressure Sensor for a Ventricular Assist Device.” Micromachines vol. 7, 8 135. 8 Aug. 2016, doi:10.3390/mi7080135.

G. E. Loeb, R. A. Peck, W. H. Moore, and K. Hood. Bion system for distributed nerual prosthetic interfaces. Medical Engineering and Physics, 23(1):9-18, January 2001.

U.S. Pat. 4,753,221 to Kensey et al. (Jun. 28, 1988).

U.S. Pat. 5,749,855 to Reitan (May 12, 1998).

U.S. Pat. 7,005,935 to Moore (Feb. 28, 2006) for “Switched reactance modulated E-class oscillator”.

U.S. Pat. 7,177,690 to Woods et al. (Feb. 13, 2007) for “Implantable system having rechargeable battery indicator”.

U.S. Pat. 7,437,193 to Parramon et al. (Oct. 14, 2008) for “Microstimulator employing improved recharging reporting and telemetry techniques”.

U.S. Pat. 7,599,743 to Hassler, Jr. et al. (Oct. 9, 2009) for “Low Frequency Transcutaneous Energy Transfer to Implanted Medical Device”.

U.S. Pat. 8,617,239 to Reitan (Dec. 31, 2013) for “Catheter Pump”.

U.S. Pat. 8,727,959 to Reitan et al. (May 20, 2014) for “Catheter Pump for Circulatory Support”.

U.S. Pat. 10,179,197 to Kaiser et al. (Jan. 15, 2019) for “Catheter Pump with a Pump Head for Insertion into the Aorta”.

U.S. Design Pat. 811,588 to Kaiser et al. (Feb. 27, 2018) for “Cage for Catheter Pump”.

U.S. Pat. Application 20200023158 A1 to Epple (Jan. 23, 2020) for “Flushing System”.

U.S. Pat. Application 20200000988 A1 to Epple (Jan. 2, 2020) for “Catheter pump comprising drive unit and catheter”.

U.S. Pat. Application 20200023109 A1 to Epple (Jan. 23, 2020) for “Catheter pump having a pump head for introducing into the arterial vasculature”.

WO 1994005347 A1 to Reitan (Mar. 17, 2994) for “Catheter Pump”.

U.S. Pat. Application 20200023113 A1 to Epple et al. (Jan. 23, 2020) for “Catheter pump with drive unit and catheter”.

U.S. Pat. Application 20020087204 A1 to Kung et al. (Jan. 4, 2001) for “Flexible transcutaneous energy transfer (TET) primary coil”.

U.S. Pat. Application 20210077687 A1 to Leonhardt (Mar. 18, 2021) for “Circulatory Assist Pump”.

International Patent Publication WO 2019/183247 A1 (Sep. 26, 2019) for “Circulatory Assist Pump”.

What is claimed is:

1. A system for a circulatory assist pump, the system comprising:
a wireless circulatory assist pump comprising:
a stent cage of a size and shape to allow a highly open flow when placed within a subject’s aorta;
at least one impeller encaged by the stent cage; and
a wireless charging circuit; and
an abdominal belt or vest comprising a coil of wire configured to extend circumferentially around a patient’s abdomen or chest and generate electromagnetic waves to provide wireless power to the wireless charging circuit.

2. The system of claim 1, wherein the abdominal belt is configured to generate electromagnetic waves at a frequency between about 50 kHz and about 300 kHz.

3. The system of claim 1, wherein the abdominal belt is configured to generate electromagnetic waves at a frequency between about 100 kHz and about 150 kHz.

4. The system of claim 1, wherein the abdominal belt is configured to generate electromagnetic waves at a frequency of about 125.3 kHz.

5. The system of claim 1, wherein the wireless charging circuit comprises a dual-coil receiver.

6. The system of claim 5, wherein the dual-coil receiver comprises a first coil and a second coil, the second coil separated from the first coil by a dielectric material.

7. The system of claim 6, wherein the dielectric material comprises an air gap.

8. The system of claim 6, wherein at least a portion of the first coil and the second coil are positioned around a magnetic core.

9. The system of claim 8, wherein at least a portion of the first coil is positioned to be exposed to electromagnetic waves generated by the coils of the abdominal belt.

10. The system of claim 9, wherein the second coil is positioned to be shielded from electromagnetic waves generated by the coils of the abdominal belt.

11. The system of claim 1, wherein the wireless circulatory assist pump further comprises:
a second impeller encaged by the stent cage.

12. A deployment and retrieval system for a circulatory assist pump, the deployment and retrieval system comprising:
a deployment catheter comprising:
an outer sheath;
an inner member; and
a plurality of fingers positioned between the outer sheath and the inner member.

13. The deployment and retrieval system of claim 12, wherein each of the plurality of fingers are configured to be biased radially outward.

14. The deployment and retrieval system of claim 13, wherein a tip of each of the plurality of fingers comprises a protrusion.

15. The deployment and retrieval system of claim 14, wherein the protrusion at the tip of each of the plurality of fingers is positioned to engage and hold an end of a circulatory assist pump when the outer sheath is deployed over the plurality of fingers and to disengage and release the end of a circulatory assist pump when the outer sheath is retracted from over the plurality of fingers.

16. The deployment and retrieval system of claim 12, further comprising:
a retrieval catheter comprising:
an outer sheath;
an inner member;
a plurality of fingers positioned between the outer sheath and the inner member; and
a magnetic portion positioned at an end of the inner member configured to mate with the end of the circulatory assist pump when positioned within proximity.

17. A method of deploying and retrieving a wireless circulatory assist pump, the method comprising:
positioning an end of a wireless circulatory assist pump adjacent an inner member of a deployment catheter;
sliding an outer sheath of the deployment catheter over a plurality of fingers to cause the fingers to engage and hold the end of the wireless circulatory assist pump;
positioning the wireless circulatory assist pump within a patient;
retracting the outer sheath of the deployment catheter from over the plurality of fingers to cause the fingers to disengage and release the end of the wireless circulatory assist pump;and
removing the deployment catheter from the patient.

18. The method according to claim 17, further comprising:
positioning a magnet located at an end of a retrieval catheter adjacent the end of the wireless circulatory assist pump to couple the magnet to the end of the wireless circulatory assist pump;
sliding an outer sheath of the retrieval catheter over a plurality of fingers of the retrieval catheter to cause the fingers of the retrieval catheter to engage and hold the end of the wireless circulatory assist pump;and
removing the wireless circulatory assist pump from the patient with the retrieval catheter.

19. A wireless mechanical circulatory assist device (MCAD) characterized in having a brushless direct current (BLDC) motor.

20. The wireless MCAD of claim 19, wherein the BLDC motor and telemetry of the MCAD are controlled by at least one application-specific integrated circuit (“ASIC”) chip.
